(19) United States
(12) Patent Application Publication
(10) Pub. No.: US 2009/0112125 A1
(43) Pub. Date: Apr. 30, 2009

(54) Integrated blood sampling and testing device and method of use thereof

(75) Inventor: Nili TAMIR, Zihron Yaakov (IL)

Correspondence Address: HOUSTON ELISEEVA, 4 MILITIA DRIVE, SUITE 4, LEXINGTON, MA, 02421, US

(73) Assignee: RAPIDX LTD., Tel Aviv (IL)

(21) Appl. No.: 12/348,973

(22) Filed: Jan. 6, 2009

Related U.S. Application Data
(63) Continuation of application No. PCT/IL2007/000856, filed on Jul. 8, 2007.

(51) Int. Cl. A61B 5/151

(52) U.S. Cl. 600/583

(57) ABSTRACT

The present invention discloses an integrated sampling and testing device for sampling and testing blood from a human finger which comprises clamping means for temporarily clamping a finger below the tip of said finger, a lancet for piercing said finger tip while said finger is held by said clamping means testing means for performing at least one test on blood drawn from said finger, wherein said testing means is aligned with said lancet such that blood drawn from said finger immediately contacts said testing means after being drawn. A method for sampling and testing human blood from a fingertip comprises the steps of: providing an integrated sampling and testing device for sampling and testing blood from a human finger, opening radially-expandable clamping member and inserting a finger therein, closing said radially-expandable clamping member around said finger such that pressure is applied to said finger so as to create a region of increased blood volume and/or pressure at the tip of said finger, piercing said finger using said lancet, ensuring that sufficient blood has been drawn and, subsequently, opening said radially-expandable clamping member so to allow removal of finger, optionally, transferring at least one reagent to said test strip using said reagent transferring means, and waiting and viewing test results.

### RELATED APPLICATIONS

This application is a Continuation of PCT application serial number PCT/IL2007/000856, filed on Jul. 8, 2007, which claims the benefit under 35 USC 119(e) of U.S. Provisional Application No. 60/818,541, filed on Jul. 6, 2006, both of which are incorporated herein by reference in their entirety.

### FIELD OF THE INVENTION

The present invention relates generally to the field of blood sampling and testing devices. More specifically, the present invention relates to an integrated device for sampling and testing blood and to a method for use thereof.

### BACKGROUND OF THE INVENTION

Diagnostic kits have become indispensable tools for certain populations. Diabetics for instance rely on routine and frequent tests of blood glucose in order to intelligently regulate their sugar intake. The purpose of monitoring the blood glucose concentration level is to take corrective action based upon whether the level is too high or too low, in order to bring the level back within a normal range. Failure to take corrective action can have serious implications; when blood glucose levels drop too low, a condition known as hypoglycemia occurs wherein a person can become nervous, shaky, and confused. That person's judgment may become impaired and he or she may eventually pass out. A person can also become very ill if their blood glucose level becomes too high, a condition known as hyperglycemia. Both conditions, hypoglycemia and hyperglycemia, are potentially life-threatening emergencies; thus diabetics have to rely on portable diagnostic kits for self-monitoring. Home pregnancy kits have become a standard home test, allowing private, quick and easy testing, at home, by oneself. Similarly home testing kits exist for cholesterol, ovulation, blood thickness, etc. Often these tests are based on color changes occurring on absorbent surfaces that have been impregnated with the appropriate reagents and are exposed by the user to their blood or urine. Alternatively electrical changes in said reagents upon exposure to body fluids may be detected by an electrochemical system.

US Patent application 20052401 19 discloses a blood glucose meter with an integral lancet device such that lancing and testing processes are positioned sufficiently close in proximity to allow a virtually continuous short motion by the user to achieve the functions of each. This device has an integral lancet (finger picker) and a plurality of test strips housed within the hollow body of the unit. It is not disposable, but rather is essentially interconnected with a computer that interfaces with an integral reader that determines the test results, saving them in a database.

German patent 10,315,396 teaches an automatic blood sugar monitor and warning system, for hypo- and hyperglycemia (low and high blood sugar level). The invention has a finger stall containing an electromagnetically activated lancet, and test strips impregnated with a glucose enzyme linked to a control. As in the case of US Patent application 2005240119, this unit is not disposable. It is intended for constant wear, and is equipped with a computer for storage and retrieval of test results.

Similarly, German patent 4,234,553 introduces an instrument for measuring blood glucose content comprising a storage container with test strips, color scale and lancet with exchangeable needle.

In the above-mentioned inventions, blood is not conveyed automatically to the test strip but rather must be applied by the user in a separate step. The device is not disposable, but rather is intended for repeated use, being equipped with a plurality of test strips. All of the above inventions, furthermore, suffer from the safety issue that arises from requiring the user to convey blood that is slowly seeping from the lanced finger onto a test strip. This blood flows at some uncontrolled rate and is subject to various jostling as the user attempts to swipe his or her finger upon a test strip, allowing for the possibility that drops of blood will be spilled. Blood spillage constitutes a health risk due to the possibility of contaminated blood and is therefore to be avoided whenever possible.

New-Zealand patent 526,334 presents a sensor with integrated lancet for monitoring blood by calorimetric or electrochemical test method. This device has the advantage that the drawing of blood and sensing thereof are performed at nearly the same location, eliminating the need to adsorb blood onto a test strip after lancing in a separate step. This device is again not disposable but rather intended for repeated use.

In the light of the above, there is a need for blood sampling and testing device that is capable of performing both blood drawing and blood testing in a continuous, safe, and quick manner.

### SUMMARY OF THE INVENTION

It is thus the primary object of the present invention to provide an integrated, “all-in-one” blood sampling and testing device that is easy to use, quick, and reliable.

It is also an object of the present invention to provide a blood sampling and testing device that may be manufactured for use for a variety of different types of blood testing.

The device of the present invention provides rapid medical diagnostic tests based on whole blood. It takes the shape of a relatively small unit with a recess that fits a person's finger.

In one preferred embodiment, the test is accomplished by piercing the skin with a lancet integrated into the body of the unit. Pulling back a lever (the Operating button’) activates the spring-loaded lancet, which pierces the skin and then retracts immediately. The same lever pushes a ‘pressing element’, a rubber ring, a clamp or any other pressure-generating element, preferably at least partially made of solid plastic or rubber; forceful acoustic wave, energized light beam etc, against the finger that increases the blood pressure in the finger, ensuring a sufficient but small amount of blood leaves the finger. This blood is immediately absorbed by a capillary sponge that conducts the blood onto a testing strip impregnated with blood-testing reagents of various sorts. Additionally, this capillary sponge may act as a reservoir to collect a predetermined volume of blood required for the performance of the diagnostic test. The testing strip provides the test result by way of a color change that indicates a positive or negative test result.

In another preferred embodiment of the present invention, the device comprises a small disposable unit equipped with a recess to fit a human finger; a spring-loaded lancet that pierces the finger and immediately retracts; a capillary sponge into which a predetermined measure blood, e.g., 1 to 100 micro liter, from the finger is absorbed; a testing strip impregnated with blood-testing reagents of various sorts onto which blood from said capillary sponge is conveyed by means of capillary action; a results-viewing window in which the results of the blood test(s) is/are displayed by means of color changes in said reagents of said testing strip; a pressing element (e.g., a sponge) that presses against the finger to increase local blood pressure ensuring extraction of blood; and an operating button in mechanical communication with the lancet and the pressing element such that pushing the operating button increases blood pressure gradually and activates the lancet at the end of the operating button's travel.

The present invention relates to an integrated sampling and testing device for sampling and testing blood from a human finger, comprising a clamping means for temporarily clamping a finger below the tip of the finger, so as to increase the volume of blood at the finger tip and thereby facilitate drawing of blood from said finger; a lancet for piercing the finger tip while the finger is held by said clamping means; and, a testing means for performing at least one test on blood drawn from the finger.

The testing means is aligned with the lancet such that blood drawn from the finger immediately contacts the testing means after being drawn from the finger tip. Thus, the user is provided with a single device for easily and effectively drawing and testing blood. The user does not need to perform any steps in order to transfer blood from the lancet to the testing means. Rather, because of the arrangement and configuration of the lancet and the testing means, blood that is extracted/withdrawn at the finger tip following piercing of the skin is conveyed directly onto the testing means.

In one preferred embodiment, the testing means are positioned at least partially in between the lancet and the clamping means, the testing means includes an opening for allowing the piercing means of the lancet to pass therethrough at the time of pricking of the finger. Thus, the blood droplets drip directly onto the testing means. Additionally or alternatively, a capillary sponge embedded in the lancet adsorbs the blood so that the blood travels by capillary action to the testing means.

Preferably, the testing means comprises a test strip, as are well-known in the art. The test strip preferably includes a housing and a capillary sponge located inside of the housing. In some preferred embodiments of the present invention, the housing is provided with a blood volume window for indicating when sufficient blood has been drawn for the particular test, and a test results window for indicating the results of the test. The results window preferably includes a control indicator and a test indicator. The test indicator may include any number of lines, such as between one and three. The results may be quantitative, qualitative, or semi-qualitative.

In some preferred embodiments, reagent transferring means are provided for transferring a predetermined amount of at least one reagent to the testing means after blood has been drawn from the finger. The reagent transferring means preferably includes at least one reagent and at least one container for holding a reagent therein. A syringe-like piston is provided for pushing the reagent out of an orifice located at the end of the reagent container. It will be appreciated that other reagent transferring means could also be employed for directly conveying at least one reagent to the testing means. It will also be appreciated that in some tests, a reagent is not required whereas in other tests, one or more reagent may need to be added in order for the test to be properly performed.

Preferably, the lancet comprises piercing means, and a lancet housing for housing the piercing means. The lancet housing also comprises an orifice located at the top portion thereof for allowing the piercing means to temporarily protrude from the housing so as to enable finger piercing. Many varieties of lancets are known in the art which may be employed. The piercing means may be, for example, a needle or a small scalpel.

According to preferred embodiments of the present invention, the lancet housing further comprises a lateral opening positioned directly below the top portion thereof for receiving at least a portion of the test strip. In one preferred embodiment, the test strip has an opening therein for allowing the piercing means to pass therethrough at the time of blood sampling. In this preferred embodiment, blood drawn from the finger tip using the piercing means automatically drips back down into the lancet orifice and onto the test strip.

Alternatively, the lancet is provided with a capillary sponge and optionally, a pressing element, that serves to apply mild pressure to the finger following piercing such that a sufficient amount of blood becomes absorbed by the capillary sponge and conveyed to the testing means in communication with the capillary sponge.

According to preferred embodiments of the present invention, the clamping means comprises a clip mechanism having a proximal end and a distal end. The proximal end includes a radially-expandable clamping member adapted for accommodating the end of a finger therein. The distal end includes a handle for actuating the radially-expandable clamping member such that the clamping member can be radially expanded and contracted around the finger for increasing local pressure thereby facilitating blood withdrawal following finger piercing. When contracted around the finger, the clamping member applies pressure to the finger such that the region of the finger which is adjacent to the clamping finger contains a high volume of blood. It will be appreciated that the clamping member is adapted for preventing backflow of blood to the body. This makes drawing of blood easier, since it makes it unnecessary to apply additional pressure to ensure that blood flows from the finger tip after piercing. Preferably, the radially-expandable clamping member comprises a flexible tourniquet having the configuration of a ring or a sleeve. It will be appreciated that other configurations are also possible. Any suitable elastic or other pressure generating material or mechanism may be used for the clamping member.

Any suitable mechanism may be employed for providing radial expansion of the clamping member. In one embodiment, for example, the clamping member is connected to the handle by three connection members equally-spaced around the circumference of the clamping member. When the handle is actuated, the connection members are pulled outward, thereby causing the clamping member to expand radially. Preferably, the handle includes two opposing hand grips and the proximal and distal end are hinged by at least one springed joint. In this manner, pressing the hand grips causes the clamping member to open to allow the user to insert the finger. Releasing the grip on the hand grips causes the clamping member to close around the finger. It is appreciated that the use of such a clamping member allows for blood sampling from fingers of a wide range of sizes.

It is appreciated that the device of the present invention may be manufactured for one-time usage only, after which it is disposed of. Alternatively, the device of the present invention may be manufactured such that only the test strip is disposed of after each use.

The present invention also relates to an integrated sampling and testing device for sampling and testing blood from a human finger. The device includes a lancet for piercing the tip of a finger, and testing means for performing at least one test on blood drawn from the finger. The testing means is aligned with the lancet such that blood drawn from the finger immediately contacts the testing means after being drawn from the finger tip. Thus, the user is provided with a single device for easily and effectively withdrawing and testing blood. The user does not need to perform any steps in order to transfer blood from the lancet to the testing means. Rather, because of the arrangement and configuration of the lancet and the testing means, blood that is produced at the finger tip following piercing of the skin is conveyed directly onto the test strip. In one scenario, the blood simply drips onto the test strip by means of surface tension or gravity. In another scenario, a capillary sponge embedded in the lancet adsorbs the blood so that the blood travels by capillary action to the testing means. In yet another scenario, suction is applied to facilitate blood transfer to the test strip.

According to preferred embodiments of the present invention, the testing means comprises a test strip. The test strip preferably includes housing and a capillary sponge located inside of the housing. In some preferred embodiments of the present invention, the housing is provided with a blood volume window for indicating when sufficient blood has been drawn for the particular test, and a test results window for indicating the results of the test. The results window preferably includes a control indicator and a test indicator. The test indicator may include any number of indication lines, such as between one and three. The results may be quantitative, qualitative, or semi-qualitative.

Further according to preferred embodiments of the present invention, the lancet housing further comprises a lateral opening positioned directly below the top portion of the housing for receiving at least a portion of the test strip. The test strip has an opening therein for allowing the piercing means to pass therethrough at the time of blood sampling, such that blood drawn from the finger tip using the piercing means automatically drips down onto the test strip.

The present invention also relates to a clamping unit for use in combination with a blood sampling device, for increasing the blood volume and/or pressure at the finger tip and thereby facilitating blood sampling. The clamping unit includes a clip mechanism having a proximal end and a distal end. The proximal end comprises a radially-expandable clamping member adapted for receiving the end of a finger therein, and the distal end comprises a handle for actuating a radially-expandable clamping member, such that when the handle is pressed by the user, the clamping member opens to allow insertion of the finger. When the handle is released, the clamping member closes so as to apply pressure onto the finger, thereby increasing blood volume and/or pressure at the finger tip.

Preferably, the radially-expandable clamping member comprises a flexible tourniquet having a configuration of a ring or a sleeve.

The present invention further relates to a method for sampling and testing human blood from a fingertip, comprising steps selected from the following:
(a) providing an integrated sampling and testing device for sampling and testing blood from a human finger, the device including:
   (i) clamping means for temporarily clamping a finger below the tip of the finger, so as to increase the volume and/or pressure of blood at the finger tip and thereby facilitate drawing of blood from the finger, the clamping unit/member comprising a radially-expandable clamping member and a handle for opening and closing the radially-expandable clamping member;
   (ii) a lancet for piercing the finger tip while the finger is held by the clamping member;
   (iii) a test strip for performing at least one test on blood drawn from the finger, wherein the test strip is aligned with the lancet such that blood drawn from the finger immediately contacts the test strip after being drawn, and;
   (iv) reagent transferring means for transferring a predetermined amount of at least one reagent to the test strip after, or concomitant with, blood withdrawal from the finger;
(b) opening the radially-expandable clamping member and inserting a finger therein;
(c) closing the radially-expandable clamping member around the finger such that pressure is applied to the finger so as to create a region of increased blood volume and/or pressure at the tip of said finger;
(d) piercing the finger using the lancet;
(e) ensuring that sufficient blood has been drawn and, subsequently, opening the radially-expandable clamping member to allow removal of finger;
(f) transferring at least one reagent to the test strip using the reagent transferring means, and;
(g) waiting and viewing/reading test results.

In some preferred embodiments, prior to step (d) of piercing the finger using the lancet, a safety stopper is removed from the lancet.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 schematically presents a compact whole-blood sampling and testing device according to one main embodiment of the present invention;

FIG. 2 schematically presents an overall view of the device according to one specific embodiment;

FIG. 3 schematically presents a cutaway view of the compact whole-blood sampling and testing device according to one embodiment of the present invention in its most simplified presentation, showing the various elements integral to its operation;

FIGS. 4a-4f schematically presents the method of operation of the compact whole-blood sampling and testing device according to one embodiment of the present invention in its most simplified presentation, showing the various stages involved in the operation of the device;

FIG. 5 schematically presents an isometric view of a second main preferred embodiment of the present invention;

FIG. 6 schematically presents a cross-sectional side view of the device of FIG. 5;

FIG. 7 schematically presents an isometric view, taken from the bottom, of the device of FIG. 5; and

FIG. 8 schematically presents an isometric view of third main preferred embodiment of the present invention.

### DETAILED DESCRIPTION OF THE PREFERRED EMBODIMENTS

The following description is provided, alongside all chapters of the present invention, so as to enable any person skilled in the art to make use of the invention and sets forth the best modes contemplated by the inventor of carrying out this invention. Various modifications, however, will remain apparent to those skilled in the art, since the generic principles of the present invention have been defined specifically to provide a compact whole-blood sampling and testing device.

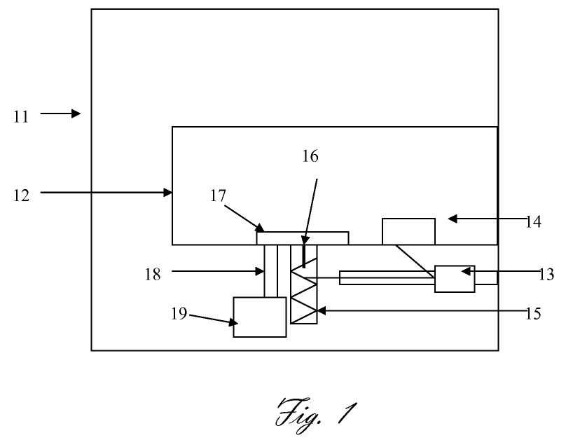

Reference is now made generally to FIGS. 1-4, and to a first main preferred embodiment of the present invention.

In a preferred embodiment of the present invention, the device includes a recess, groove or any other form of placement that fits a person's finger, and is equipped with a lancet, a pressing element, a capillary sponge, and calorimetric, fluorimetric, electronic or other testing strip. The test is accomplished by piercing the skin with the lancet, which is integrated into the body of the unit. Pulling back a lever (the Operating button’) activates the spring-loaded lancet, which pierces the skin and then retracts immediately. The same lever pushes a ‘pressing element’ against the finger that increases the blood pressure in the finger, ensuring a sufficient but small amount of blood leaves the finger. This blood is absorbed by a capillary sponge that may act according a specific embodiment of the invention as a reservoir to collect a predetermined volume of blood required for the test.

The capillary sponge may further be impregnated with, coated by or composed of blood-testing reagents of any sorts. Alternatively or additionally, the capillary sponge further directs blood onto a testing strip, optionally impregnated with blood-testing reagents of various sorts. The testing strip provides the test result by way of a color change that indicates a positive or negative test result. The device is small, lightweight, and inexpensive, and is intended to be a disposable item that is used and then discarded.

It is in the scope of the present invention wherein the capillary sponge serves as reservoir for collecting the volume of blood required for the test. Nevertheless, another blood reservoir or reservoirs are possible.

The device is especially useful for diagnosing health conditions based on determination of concentrations, conductivity, viscosity, and the like of chemical and/or biological factors selected in a non-limiting manner from cholesterol, for assessing risk of heart disease; glucose, for monitoring diabetes; the presence of illegal drugs and drugs of abuse; hCG, to screen for pregnancy; HIV-antibody, for determining HIV infection; prothrombin time, for monitoring blood thinning and clotting; fecal occult blood, to screen for colorectal cancer; and luteinizing hormone, to predict ovulation or any combination thereof.

The unit, requiring only a simple calorimetric chemical reaction for its operation, requires no electricity or computer. However, other modes of detection, such as fluorimetric, electronic, or any other sensory-discernable signal are also within the scope of this application. It can be used anywhere, being lightweight, portable, and disposable. It provides rapid and accurate diagnosis based on exhaustively investigated biological, chemical or physical reactions with whole blood that have been perfected by the medical industry. Blood spillage is entirely avoided since the blood is conveyed immediately after it is drawn onto the capillary sponge. Similarly, the entire volume of blood required is minimized, eliminating the health hazard of spillage and the psychological factors involved with the sight of blood, in addition to decreasing the physical pain associated with drawing a larger amount of blood.

It is acknowledge in this respect that according to another embodiment of the present invention, the ‘pressing element’ provides multiple effects, e.g., applying required pressure for enabling blood to be extracted, acting to reduce the pain associated with lancet piercing, etc. Blood is possibly extracted by the patient himself in a semi-automatic manner, hence the possibility of transferring blood-borne pathogens to Medicare personnel is reduced or even entirely eliminated.

FIG. 1 presents a schematic illustration of the device according to one embodiment of the present invention, wherein 11 denotes the Blood sampling and testing device, side view schematic; 12 denotes sampling cavity (recess for finger or other body part); 13 denotes operating button; 14 denotes pressing element; 15 denotes spring; 16 denotes lancet; 17 denotes capillary sponge; 18 denotes test strip; and 19 is denoted for an optional results window

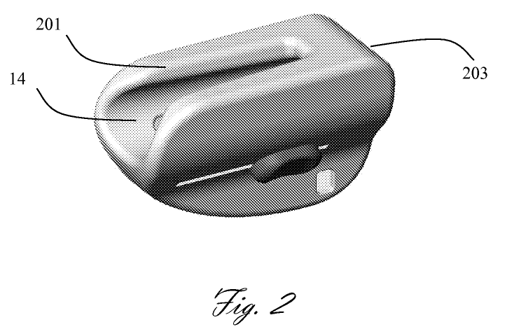

Reference is now made to FIG. 2, presenting a perspective view of a device according to another specific embodiment of the present invention, showing a receptacle for the finger, an operating button, and results window (small square at lower right). This embodiment is especially adapted to immobilize the finger of a patient for blood sampling. Other designs are adapted to accommodate, glue, immobilize, entrap, clasp, fasten or otherwise reversibly interconnect a superficial body portion, selected for example and in a non-limiting manner to tubular body portions (fingers, arms, legs, neck etc) or surfaces (such as toddlers or infant's foot, ear etc). Those entrapping means (201), are possibly facilitating a cavity (202), so as a body portions as defined above are at-least partially accommodated, here for example a finger is inserted up to a stop (203). Other designs of the entrapping means (201) are possible so as its size and shape are adapted to fit the size and shape of the sampled body organ.

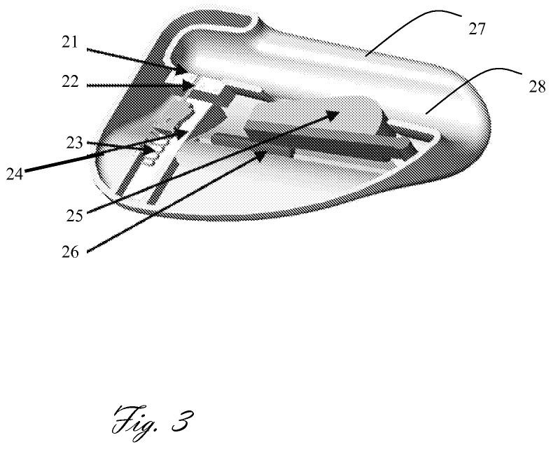

Reference is now made to FIG. 3, presenting a cross section of the device according to the specific embodiment as defined and described above. At least one pressing element (25) perturbs sampling cavity (28) enveloped by entrapping means (27), possibly, yet not exclusively, made of thermosetting polymers, silicones, elastic carbohydrates, rubber-like material, etc. As the operating button (26) is pressed, the pressing element (25) presses the finger or other body part to be tested to increase the local blood pressure. At the end of the operating button's (26) travel, the spring (23) activates the lancet (22) which then pierces the skin. Blood leaks out of the skin to be absorbed by the capillary sponge (21) from where it is conveyed by capillary action to the testing strip (24). The capillary sponge (21) has a measurable capacity to absorb sampled blood, for example, about 0.05 to about 5 ml. Capillary sponge (21) and/or tester strip (24) are either passive (absorbing only) or active members. An active sponge is possibly adapted to treat the sampled organ. Hence for example, said sponge is possibly immersed, doped, impregnated, socked, coated or otherwise treated to contain Heparin or other compositions that inhibit blood coagulation, blood coagulators, such as calcium salts or factors, antibiotics, sterilizers such as quaternary ammonium salts or H₂O₂; indicators, such as HIV or glucose indicators; and other additives, selected in a non-limiting manner from colorants, biocides, perfumes, fillers, emulsifiers, super absorbents, especially acrylic derivatives, etc. The capillary sponge (21) may be at least partially made of organic matter, such as cotton or polymers, and/or inorganic matter, such as bentonite etc.

It is acknowledged in this respect that any of sponge (21) and/or tester strip (24) is possibly adapted to provide online or offline diagnosis. Moreover, this diagnosis may be either in situ, e.g., being provided by color change etc., or otherwise. Hence for example, the members are possibly interconnected to a remote displayer, via for example RFID or other disposable and cost effective wireless means.

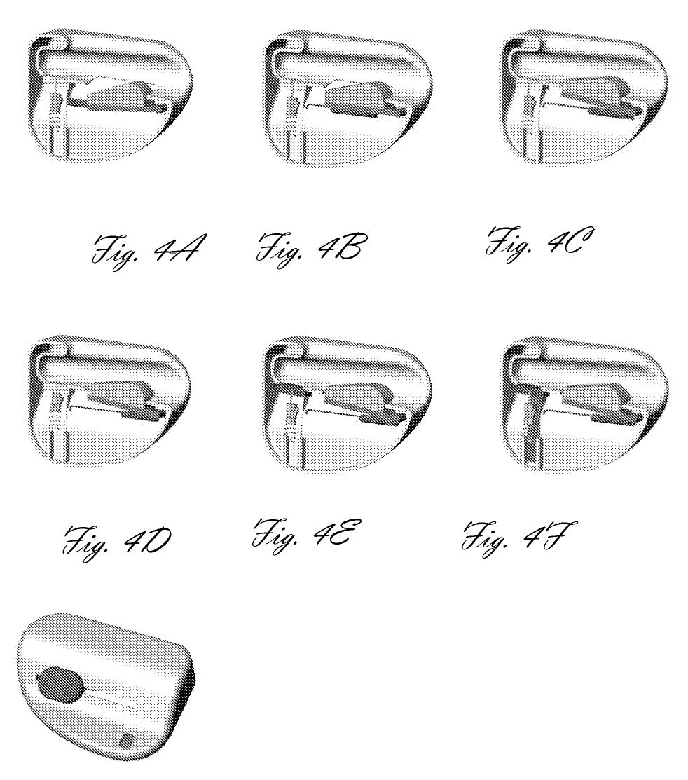

Reference is now made to FIGS. 4A-4G, presenting possible operating steps selected in a non-limiting manner from a. operating button retracted; b. button moved forward; c. button moved further forward, increasing blood pressure; d. lancet released, piercing skin, and retracted immediately; e. blood adsorbed onto capillary sponge; f. blood conveyed to testing strip by capillary action automatically; g. test results appear in test window through calorimetric reaction. Hence for example, and still in a non-limiting manner, a mode of operation according to yet one embodiment of the present invention may be divided into several sub-stages, explained in the following list:

Stage 1 (FIG. 4a)
a. Operating button is all the way back;
b. Lancet is primed;
c. Pressing element is at its lowest position; and,
d. Finger is entered into the device until fingertip touches the capillary sponge.

Stage 2 (FIG. 4b)
a. Moving the operating button forward;
b. Pressing element moves up;
c. Finger starts being pressed; and,
d. Blood flow is being restricted.

Stage 3 (FIG. 4c)
a. Moving the operating button even further; and
b. Blood pressure in the fingertip increases.

Stage 4 (FIG. 4d)
a. Moving the operating button all the way;
b. Lancet is released and pierces the skin; and,
c. Lancet retracts immediately.

Stage 5 (FIG. 4e)
a. Blood is withdrawn from pierced Fingertip to capillary sponge;
b. The capillary sponge serves as reservoir for collecting the amount of blood required for the test; and,
c. Finger may now be removed from the device.

Stage 6 (FIG. 4f)
a. Blood moves by capillary flow from sponge to tester strip; and,
b. Specific reagents impregnated in the sponge and tester strip react with blood components.

Stage 7 (FIG. 4g)
a. Test result will appear in Results window.

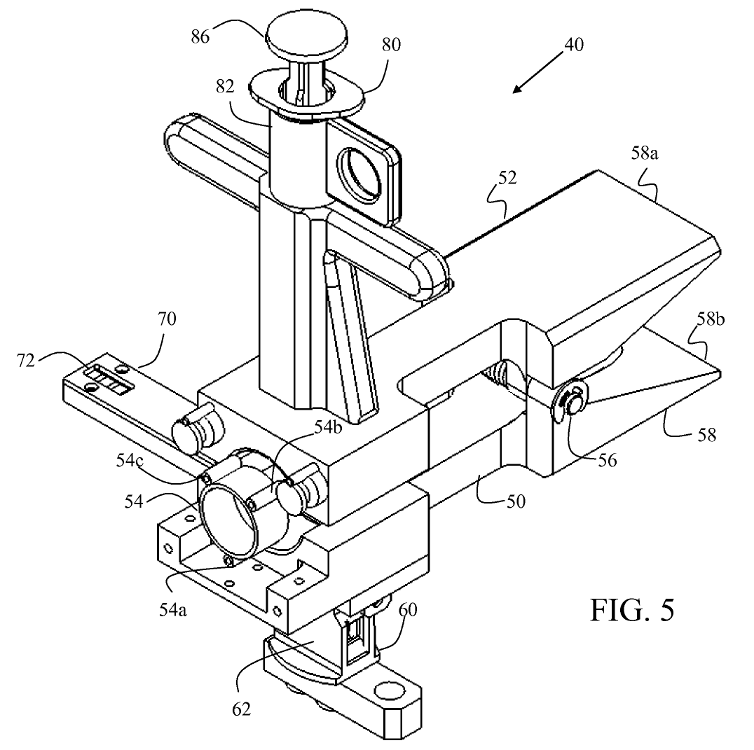

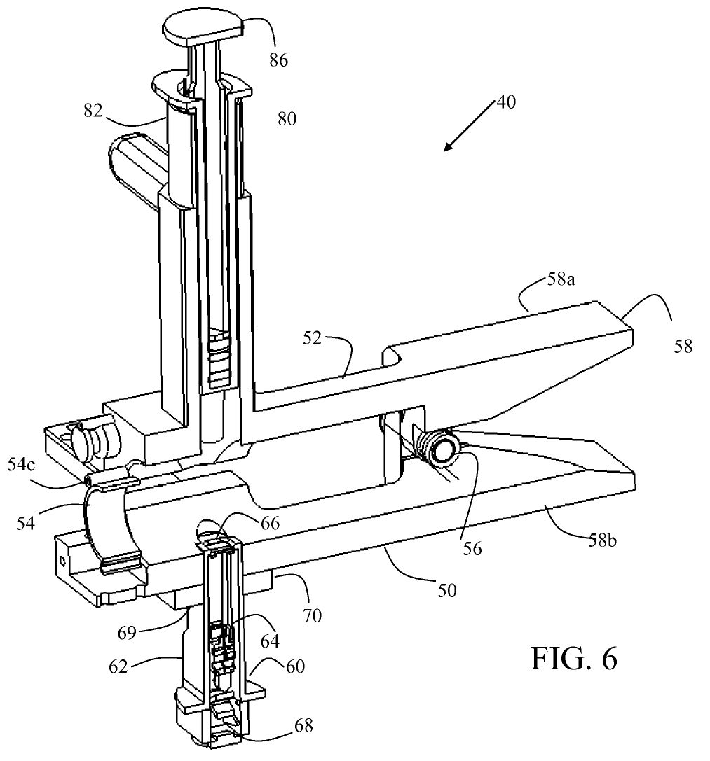

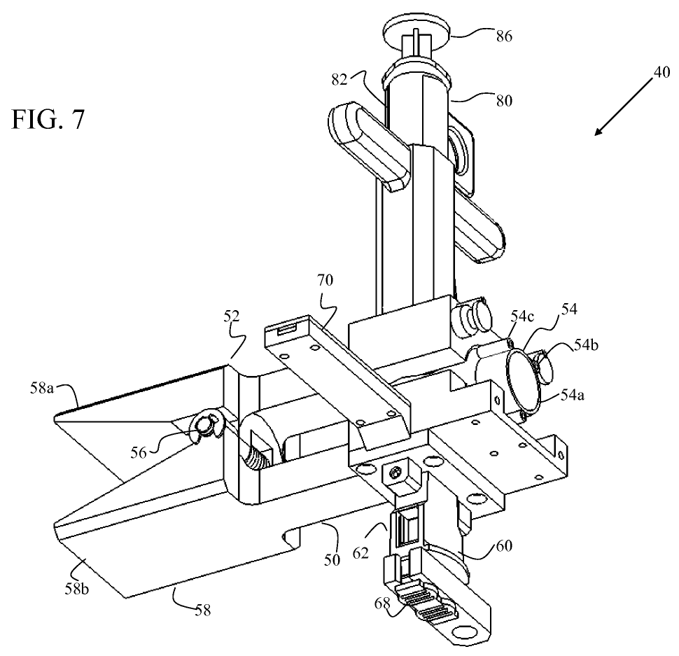

Reference will now be made to FIGS. 5-7, and to a second main preferred embodiment of the present invention.

Referring first to FIG. 5, blood sampling device (40) includes four main components: clamping means (50) adapted for accommodating the end of a finger therein and for clamping the finger so as to produce an area of increased blood volume and/or pressure to facilitate drawing of blood; a lancet (60) for piercing the finger using a scalpel or needle while the finger is held by clamping means (50); a test strip (70) for performing at least one test on blood drawn from the finger, and; reagent transferring means (80) for transferring at least one reagent to test strip (70). It will be appreciated that in other embodiments, other testing means may be employed in addition to, or instead of, a test strip. Furthermore, test strip (70) may be adapted for performing any known biological test, such as, but not limited to, pregnancy, ovulation, cholesterol, blood thickness, or sugar.

It is noted that the preferable orientation of the device is as follows: clamping means (50) and test strip (70) are substantially in the same plane, while reagent transferring means (80) and lancet (60) are positioned substantially perpendicular to clamping means (50) and test strip (70), with transferring means (80) positioned above the plane of clamping means (50)/test strip (70) and lancet (60) positioned below clamping means (50)/test strip (70). Preferably, clamping means (50) includes a connection for attachment of test strip (70) to one side of clamping means (50). The test strip is disposed of and replaced after each usage. Alternatively, the entire device is intended for one-time-use only and is disposed of after being used.

Referring now in combination to FIGS. 5-7, clamping means (50) comprises a clip mechanism (52) having a proximal end (52a) and a distal end (52b). Proximal end (52a) includes a radially-expandable clamping member (54). The interior of clamping member (54) defines a recess for accommodating the finger. Clamping member (54) may comprise a sleeve, a ring, or any other elastic tourniquet type of member having any suitable width or thickness. It will be appreciated that clamping member (54) may be designed to accommodate a wide range of sizes of fingers. Optionally, different types of clamping members (54) may be manufactured for children and adults.

Distal end (52b) of clip mechanism (52) comprises a handle (58) having first and second hand grips (58a) (58b). Hand grips (58a) (58b) are mechanically connected to one another by spring joint (56). In the preferred embodiment illustrated, clamping member (54) expands and contracts through the use of three connection members (54a, 54b, 54c) which are equally spaced around the outer circumference of clamping member (54). Thus, when hand grips (58a) (58b) are squeezed together, this causes clamping member (54) to expand radially, so as to allow insertion or removal of the finger. When hand grips (58a) (58b) are relaxed, clamping member (54) contracts around the finger, applying pressure thereto.

Device (40) also includes a lancet (60). A variety of lancets are known in the art which could be readily adapted for use with the device of the present invention. Lancet (60) includes a lancet housing (62) and piercing means (64) contained therein, and an orifice (66) at the top of housing (62) for allowing temporary protrusion therethrough of piercing means (64) at the time of blood sampling. It noted that orifice (66) is positioned below clip mechanism (52) in a manner such that a finger held by clamping member (54) can be pierced by piercing means (64) at the region of the finger where blood volume and/or pressure is increased.

Piercing means (64) preferably comprises a needle or small scalpel, as is known in the art. Other suitable piercing means may also be used for piercing an opening in the skin in order to draw blood. In most preferred embodiments, a safety stopper (68) is also provided in order to prevent accidental or premature actuation of piercing means (64). Prior to activating piercing means (64) of lancet (60), the user should remove stopper (68).

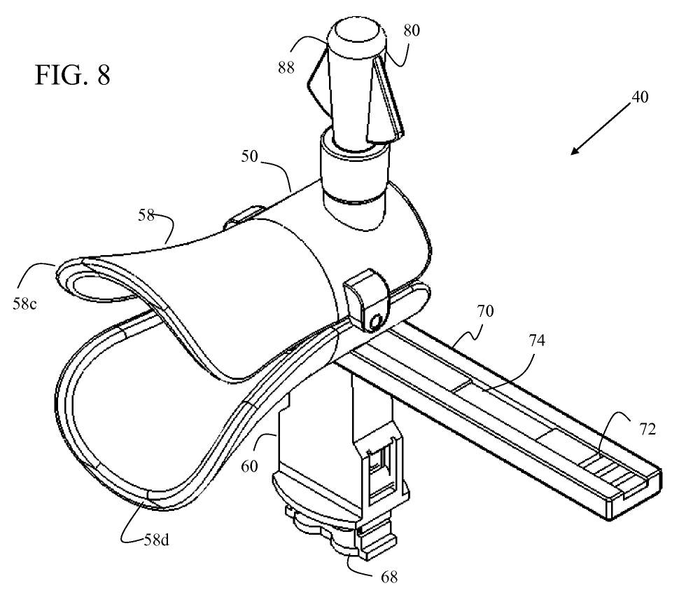

Test strip (70) may comprise any suitable test strip, as are well known in the art, for performing different tests. Preferably, a capillary sponge inside the test strip includes at least one enzymatic substrate for reacting with the blood that is sampled and providing an indication of the presence/absence, and optionally, the quantity of a specific protein or other biological factor. Alternatively or additionally, reagent transferring means (80) is employed for transferring a predetermined amount of one or more reagents to test strip (70) after blood has been sampled. Test strip (70) includes a test results window (72) for viewing the results of the test after sufficient time has elapsed. Usually, one test line in window (72) represents a control, and one or two additional test lines provide an indication of the test results. In preferred embodiments of the present invention, test strip (70) also includes a blood volume indication window (74; seen in FIG. 8). Because the device of the present invention provides for continuous blood sampling-blood testing, it is preferable to have an indication of when sufficient amount of blood has been drawn for performing the particular test. Blood volume window (74) serves this function by providing a window to the capillary sponge so that the user can see when enough blood has been drawn—an indication of this being that the blood has traveled the entire length of the sponge.

Reagent transferring means (80) preferably operates in a manner similar to a standard piston-syringe: Reagent transferring means (80) includes at least one container (82) for holding a reagent or combination of reagents therein. A piston (86) is coupled to container (82) for releasing the contents of container (82) via an opening (84) at the bottom of container (82). Opening (84) is in direct alignment with test strip (70) such that the reagent(s) are delivered directly to test strip (70).

In one preferred embodiment, test strip (70) is positioned between lancet (60) and clamping means (50). An opening is provided on test strip (70) which allows for piercing means (64) to pass therethrough to the finger at the time of blood sampling. In such a manner, the blood droplets which are released from the finger drop or otherwise flow (e.g. by means of surface tension) directly onto test strip (70).

Alternatively, as in the embodiment illustrated, lancet (60) is provided with a lateral opening (69) which holds the end of test strip (70). Test strip is provided with an opening which allows piercing means to pass therethrough to the finger at the time of blood sampling. This arrangement also allows for the blood droplets which are released from the finger to drop or flow through orifice (66) and directly onto test strip (70).

The method of operation of the device of the present invention will now be described. To operate device (40), the user first squeezes/presses hand grips (58a) (58b) of handle (58) so as to radially expand clamping member (54) to achieve a diameter large enough for the insertion of the finger. Following insertion, the user releases hand grips (58a) (58b), thereby causing clamping member (54) to constrict around the circumference of the finger. This causes the blood volume and/or pressure at the finger tip to become increased. Next, the user releases the safety stopper (68) on the bottom of lancet (60) and actuates piercing means (64) of lancet (60), causing the finger to become pricked. Because of the positioning of test strip (70) with respect to clamping means (50), blood droplets which fall from the finger drip or flow directly onto test strip (70). (In some preferred embodiments, an additional capillary sponge or other pressurizing means are provided at the top of lancet (60) serving to apply mild pressure to the finger such that blood droplets readily flow from the finger). The user may ensure that sufficient blood has been drawn by viewing blood volume indication window (74) of test strip (70). Following this step, the user again squeezes/presses hand grips (58a) (58b) so as to cause radial expansion of clamping member (54) and thus allow removal of the finger from the clamping means (50).

Optionally, the user then presses on piston (86) of reagent transferring means (80) such that the appropriate reagent(s) for the specific test are transferred to test strip (70). Next, the user waits until the results of the test appear in test results window (72).

It is appreciated that the device of the present invention provides a user with the ability to sample and test its own blood in a quick and efficient manner. However, it is also possible for the device to be operators by health care providers for testing the blood of a patient.

The embodiment illustrated in FIG. 8 is similar to that of FIGS. 5-8, with the exception that the reagent introducing means (80) and the handle (58) of clamping means (50) have different designs than that of the previous embodiment. In this case, reagent introducing means comprises a reagent feeder (88). Handle (58) comprises an upper portion (58c) and a lower portion (58d) which are gripped in a manner similar to the hand grips of the previous embodiment in order to radially expand and contract the clamping member.

While this invention has been particularly shown and described with references to preferred embodiments thereof, it will be understood by those skilled in the art that various changes in form and details may be made therein without departing from the scope of the invention encompassed by the appended claims.

What is claimed is:

1. An integrated sampling and testing device for sampling and testing blood from a human finger, comprising;
a. clamping means for temporarily clamping a finger below the tip of said finger, so as to increase the volume and/or pressure of blood at said finger tip and thereby facilitate drawing of blood from said finger;
b. a lancet for piercing said finger tip while said finger is held by said clamping means, and;
c. testing means for performing at least one test on blood drawn from said finger,
wherein said testing means is aligned with said lancet such that blood drawn from said finger immediately contacts said testing means after being drawn.

2. The integrated sampling and testing device according to claim 1, further comprising reagent transferring means for transferring a predetermined amount of at least one reagent to said testing means after blood has been drawn from said finger.

3. The integrated sampling and testing device according to claim 1, wherein said lancet comprises piercing means, and a lancet housing for housing said piercing means, and wherein said lancet housing further comprises an orifice located at the top portion thereof for allowing said piercing means to temporarily protrude from said housing so as to enable blood sampling.

4. The integrated sampling and testing device according to claim 1, wherein said testing means comprises a test strip.

5. The integrated sampling and testing device according to claim 4, wherein said lancet further includes a capillary sponge for absorbing blood and for conducting blood to said test strip.

6. The integrated sampling and testing device according to claim 4, wherein said test strip includes a housing and a capillary sponge located inside of said housing, and wherein said housing is provided with a blood volume indication window for indicating when sufficient blood has been drawn for the particular test, and a test results window for indicating the results of said test.

7. The integrated sampling and testing device according to claim 4, wherein said lancet housing further comprises a lateral opening positioned directly below said top portion for receiving at least a portion of said test strip, and wherein said test strip has a small orifice therein for allowing said piercing means to pass therethrough at the time of finger piercing, such that blood drawn from the finger tip using said piercing means automatically drips or flows onto said test strip.

8. The integrated sampling and testing device according to claim 1, wherein said clamping means comprises a clip mechanism having a proximal end and a distal end, said proximal comprising a radially-expandable clamping member, and said distal end comprising a handle for actuating said radially-expandable clamping member such that said clamping member can be radially expanded and contracted around said finger for facilitating sampling of blood.

9. The integrated sampling and testing device according to claim 8, wherein said radially-expandable clamping member comprises a flexible tourniquet having a configuration of a ring or a sleeve.

10. An integrated sampling and testing device for sampling and testing blood from a human finger, comprising a lancet for piercing the tip of a finger, and testing means for performing at least one test on blood drawn from said finger, wherein said testing means is aligned with said lancet such that blood drawn from said finger immediately contacts said testing means after being drawn.

11. The integrated sampling and testing device for sampling and testing blood from a human finger according to claim 10, wherein said testing means comprises a test strip.

12. The integrated sampling and testing device according to claim 10, wherein said test strip includes a housing and a capillary sponge located inside of said housing, and wherein said housing is provided with a blood volume indication window for indicating when sufficient blood has been drawn for the particular test, and a test results window for indicating the results of said test.

13. The integrated sampling and testing device according to claim 10, wherein said lancet housing further comprises a lateral opening positioned directly below said top portion for receiving at least a portion of said test strip, and wherein said test strip has an orifice therein for allowing said piercing means to pass therethrough at the time of finger piercing, such that blood drawn from the finger tip using said piercing means automatically drips or flows onto said test strip.

14. A clamping unit for use in combination with a blood sampling device, for increasing the blood volume and/or pressure at the finger tip and thereby facilitating blood sampling, said clamping unit comprising a clip mechanism having a proximal end and a distal end, wherein said proximal end comprises a radially-expandable clamping member adapted for receiving the end of a finger therein, and wherein said distal end comprises a handle for actuating said radially-expandable clamping member, such that when said handle is squeezed/pressed by the user, said clamping member opens to allow insertion of the finger, and when said handle is released, said clamping member closes so as to apply pressure onto the finger, thereby increasing blood volume and/or pressure at the finger tip.

15. The integrated sampling and testing device according to claim 14, wherein said radially-expandable clamping member comprises a flexible tourniquet having a configuration of a ring or a sleeve.

16. A method for sampling and testing human blood from a fingertip, comprising the steps of:
(a) providing an integrated sampling and testing device for sampling and testing blood from a human finger, the device including:
(i) clamping means for temporarily clamping a finger below the tip of said finger, so as to increase the volume and/or pressure of blood at said finger tip and thereby facilitate drawing of blood from said finger, said clamping means comprising a radially-expandable clamping member and a handle for opening and closing said radially-expandable clamping member;
(ii) a lancet for piercing said finger tip while said finger is held by said clamping member;
(iii) a test strip for performing at least one test on blood drawn from said finger, wherein said test strip is aligned with said lancet such that blood drawn from said finger immediately contacts said test strip after being drawn; and,
(iv) reagent transferring means for transferring a predetermined amount of at least one reagent to said test strip after blood has been drawn from said finger;
(b) opening said radially-expandable clamping member and inserting a finger therein;
(c) closing said radially-expandable clamping member around said finger such that pressure is applied to said finger so as to create a region of increased blood volume and/or pressure at the tip of said finger;
(d) piercing said finger using said lancet;
(e) ensuring that sufficient blood has been drawn and, subsequently, opening said radially-expandable clamping member so to allow removal of finger;
(f) optionally, transferring at least one reagent to said test strip using said reagent transferring means; and,
(g) waiting and viewing test results.
